(19) United States
(12) Patent Application Publication
(10) Pub. No.: US 2021/0086473 A1
(43) Pub. Date: Mar. 25, 2021

(54) NONWOVEN OR FABRIC ELASTICIZED WITH A MULTIPLICITY OF FIBER STRANDS IN A CLOSE PROXIMITY

(71) Applicant: A&AT LLC,, Wilmington, DE (US)

(72) Inventor: Kofi BISSAH, Newark, DE (US)

(73) Assignee: The LYCRA Company LLC, Wilmington, DE (US)

(21) Appl. No.: 16/971,787

(22) PCT Filed: Feb. 11, 2019
(86) PCT No.: PCT/US2019/017535

§ 371 Date: Aug. 21, 2020

Related U.S. Application Data
(60) Provisional application No. 62/634,222, filed on Feb. 23, 2018.

(51) Int. Cl. B32B 5/26; D04H 1/593; B32B 5/02; B32B 7/12; A61F 13/49; A61F 13/00

(52) U.S. Cl. CPC B32B 5/26; D04H 1/593; B32B 5/022; B32B 7/12; B32B 2535/00; A61F 13/00038; B32B 2307/51; B32B 2262/0207; B32B 2555/02; A61F 13/4902

(57) ABSTRACT

Disposable or reusable elasticized or stretchable nonwoven or fabric composites with multiple ends arranged in close spacing as well as methods for their production are provided.

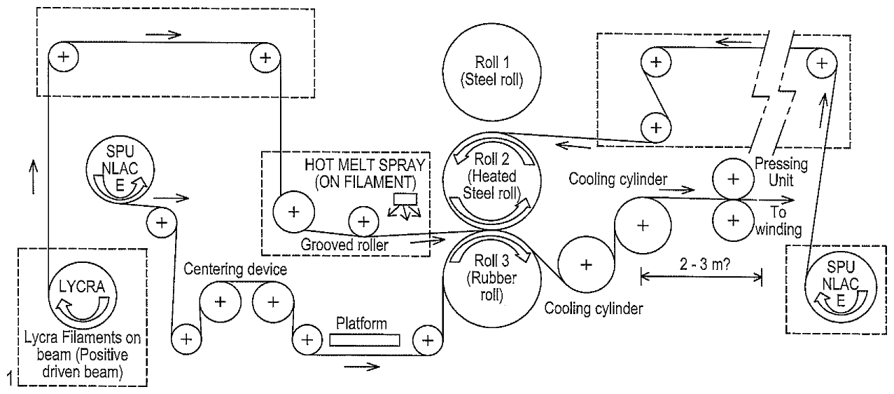

### FIELD OF THE INVENTION

The present invention relates to disposable or reusable elasticized or stretchable nonwovens or fabrics with multiple ends arranged in close spacing as well as methods for their production. These nonwovens and fabrics are useful in a variety of applications including, but not limited to, home textiles, medical components, personal and hygiene articles such as diapers and adult incontinence garments, and bandages.

### BACKGROUND OF THE INVENTION

Stretch nonwovens or elasticized fabrics are widely used for feminine hygiene, adult Incontinence, and infant and child care purposes. These nonwovens or fabrics are produced online and integrated with the diaper or adult incontinence production. However, they are limited to wide spacing and fewer ends due to the inability of the diaper or medical manufacturers to produce wide fabrics (12 inches-65 inches) with multiple fiber ends in close spacing arrangement.

U.S. Pat. No. 6,713,415 discloses a laundry-durable composite fabric, based on two non woven outer layers and pre-stretched inner layer of elastomeric fibers of at least 400 decitex and at least 8 threadlines/inch.

There is a need for disposable or reusable elasticized or stretchable nonwoven or fabric composites and methods for this production which solve problems where wider webs and offline standalone production is required.

### SUMMARY OF THE INVENTION

An aspect of the present invention is related to stretch nonwoven or elasticized fabric composites comprising two outer layers of nonwoven or fabric of substantially equal width wherein each layer has an inside surface and an outside surface with respect to the composite fabric, an inner layer of elastomeric fibers with multiple ends arranged in close spacing; and an adhesive composition bonding the outer and inner layers.

In one nonlimiting embodiment, the inner layer of elastomeric fibers comprises 10-700 ends. In one nonlimiting embodiment, elastomeric fibers of inner layer of are spaced 1.5 mm-5 mm apart.

Another aspect of the present invention relates to a process for manufacturing a stretch nonwoven or elasticized fabric composite. The process comprises placing between two layers of nonwoven or fabric an inner layer of elastomeric fibers with multiple ends arranged in close spacing. In one nonlimiting embodiment, the inner layer is under tension. In one nonlimiting embodiment, the inner layer is drafted 2× to 4×. In one nonlimiting embodiment, the inner layer is drafted 2.5× to 4×. The two layers of nonwoven or fabric and the inner layer of elastomeric fibers are then bonded by applying an adhesive composition. In one nonlimiting embodiment, the adhesive is applied to the inner layer fibers and attached to the nonwoven. In one nonlimiting embodiment, the nonwoven is free from adhesive.

In one nonlimiting embodiment, a beam arranged fiber feeding system is used to feed the inner layer of elastomeric fiber and adhesive onto the top and/or bottom nonwoven or fabric outer layers. In another nonlimiting embodiment, a multi creel fiber arranged system is used to feed the inner layer of elastomeric fiber and adhesive is applied to the inner layer fiber before attaching onto the top and/or bottom nonwoven or fabric outer layers.

In one nonlimiting embodiment of this process, the inner layer of elastomeric fibers comprises 10-700 ends. In one nonlimiting embodiment of this process, the elastomeric fiber of the inner layer is spaced 1.5 mm-5 mm apart.

Another aspect of the present invention related to articles of manufacture, at least portion of which comprises the stretch nonwoven or elasticized fabric composite disclosed herein.

### BRIEF DESCRIPTION OF THE FIGURE

The FIGURE is a diagram outlining a nonlimiting embodiment of a process for production of a disposable or reusable elasticized or stretchable nonwoven or fabric composite of the present invention.

### DETAILED DESCRIPTION OF THE INVENTION

Provided by this disclosure are disposable or reusable elasticized or stretchable nonwoven or fabric composites and methods for production of these stretchable nonwoven or fabric composites that are useful, for example, as home textiles, medical components, personal and hygiene articles such as diapers, adult incontinence garments, bandages etc and methods for theirs production.

The disposable or reusable elasticized or stretchable nonwoven or fabric composites of the present invention comprise two outer layers of nonwoven or fabric each having inside and outside surfaces. In one nonlimiting embodiment, these two outer layers are of substantially equal width.

The disposable or reusable elasticized or stretchable nonwoven or fabric composites of the present invention further comprise an inner layer of elastomeric fibers with multiple ends arranged in close spacing.

By “multiple ends”, as used herein, it is meant to include, but is not limited to about 10 to about 700 ends.

By “close spacing”, as used herein, it is meant that the elastomeric fiber is spaced 1.5 mm-5 mm apart.

In one nonlimiting embodiment, at least a portion of the elastomeric fiber comprises spandex.

In addition, the disposable or reusable elasticized or stretchable nonwoven or fabric composites of the present invention comprise an adhesive composition bonding the outer and inner layers.

Various substrates may be used as the outer layers.

In one nonlimiting embodiment, a relatively inelastic outer layer for elasticizing as described herein is used. Nonwoven substrates or “webs” are substrates having a structure of individual fibers, filaments or threads that are interlaid, but not in an identifiable, repeating manner. Nonwoven substrates can be formed by a variety of conventional processes such as, for example, meltblowing processes, spunbonding processes and bonded carded web processes. A nonlimiting example of a carded web process is spunlacing which uses hydro jets to entangle the staple fibers. Meltblown substrates or webs are those made from meltblown fibers. Meltblown fibers are formed by extruding a molten thermoplastic material through a plurality of fine, usually circular, die capillaries as molten thermoplastic material or filaments into a high velocity gas (e.g. air) stream. This attenuates the filaments of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, U.S. Pat. No. 3,849,241, which patent is incorporated herein by reference.

Spunbonded substrates or “webs” are those made from spunbonded fibers. Spunbonded fibers are small diameter fibers formed by extruding a molten thermoplastic material as filaments from a plurality of fine, usually circular, capillaries of a spinerette. The diameters of the extruded filaments are then rapidly reduced as by, for example, stretching or other well-known spun-bonding mechanisms. The production of spun-bonded nonwoven webs is illustrated, for example, in U.S. Pat. Nos. 3,692,618 and 4,340,563, both of which patents are incorporated herein by reference.

The relatively inelastic substrates can be constructed from a wide variety of materials. Suitable materials, for example, can include: polyethylene, polypropylene, polyesters such as polyethylene terephthalate, polybutane, polymethyidentene, ethylenepropylene co-polymers, polyamides, tetrablock polymers, styrenic block copolymers, polyhexamethylene adipamide, poly-(oc-caproamide), polyhexamethylenesebacamide, polyvinyls, polystyrene, polyurethanes, polytrifluorochloroethylene, ethylene vinyl acetate polymers, polyetheresters, cotton, rayon, hemp and nylon. In addition, combinations of such material types may be employed to form the relatively inelastic substrates to be elasticized herein.

Preferred substrates to be elasticized herein include structures such as polymeric spunbonded nonwoven webs. Particularly preferred are spunbonded polyolefin nonwoven webs having a basis weight of from about 10 to about 40 grams/m². More preferably such structures are polypropylene spunbonded nonwoven webs having a basis weight of from about 14 to about 25 grams/m².

The relatively inelastic substrates as hereinbefore described can be elasticized by adhesively bonding to one or more of such substrates a certain type of elastomeric polyurethane material. Such adhesive bonding to the substrate to be elasticized occurs while the polyurethane material is drafted to an elongated state.

In one nonlimiting embodiment, the elastomeric fiber of the inner layer comprises spandex.

The spandex fiber of the present invention meets the definition of “a manufactured fiber in which the fiber-forming substance is a long chain synthetic polymer comprised of at least 85% of a segmented polyurethane”. The elastic properties and the retention of the elastic properties after heat treatment of a spandex fiber are very much dependent on the content of the segment polyurethane, and the chemical composition, the micro domain structure and the polymer molecular weight of the segment polyurethane. As it has been well established, segmented polyurethanes are one family of long chain polyurethanes consisting of hard and soft segments by step polymerization of a hydroxyl-terminated polymeric glycol, a diisocyanate and a low molecular weight chain extender. Depending on the nature of the chain extender used, a dial or a diamine, the hard segment in the segmented polyurethane can be urethane or urea. The segmented polyurethanes with urea hard segments are categorized as polyurethaneureas. In general, the urea hard segment forms stronger inter-chain hydrogen bonding functioning as physical cross-link points, than the urethane hard segment. Therefore, a diamine chain extended polyurethaneurea typically has better formed crystalline hard segment domains with higher melting temperatures and better phase separation between soft segments and hard segments than a short chain dial extended polyurethane. Because of the integrity and resistivity of the urea hard segment to thermal treatment, polyurethaneurea are typically spun into fibers through a solution spinning process, either wet spinning or dry spinning. Polyurethane fibers, produced with urethane hard segments, and selected polyurethaneurea fibers may also be produced by melt spinning.

A mixture or blend of two or more segmented polyurethanes or polyurethaneureas can be used. Optionally, a mixture or blend of the segmented polyurethaneurea can also be used with another segmented polyurethane or other fiber forming polymers.

The polyurethane or polyurethaneurea is made by a two-step process. In the first step, an isocyanate-terminated urethane prepolymer is formed by reacting a polymeric glycol with a diisocyanate. Typically, the molar ratio of the diisocyanate to the glycol is controlled in a range of 150 to 2.50. If desired, catalyst can be used to assist the reaction in this prepolymerization step. In the second step, the urethane prepolymer is dissolved in a solvent such as N,N-dimethylacetamide (DMAc) and is chain extended with a short chain diamine or a mixture of diamines to form the polyurethaneurea solution. The polymer molecular weight of the polyurethanurea is controlled by small amount of mono-functional alcohol or amine, typically less than 60 milliequivalent per kilogram of the polyurethaneurea solids, added and reacted in the first step and/or in the second step. The additives can be mixed into the polymer solution at any stage after the polyurethaneurea is formed but before the solution is spun into the fiber. The total additive amount in the fiber is typically less than 10% by weight. The solid content including the additives in the polymer solution prior to spinning is typically controlled in a range of 30.0% to 40.0% by weight of the solution. The solution viscosity is typically controlled in range from 2000 to 5000 poises for optimum spinning performance. Suitable segmented polyurethane polymers can also be made in the melt, provided that the hard segment melting point is low enough Suitable polymeric glycols for the polyurethaneurea include polyether glycols, polycarbonate glycols, and polyester glycols of number average molecular weight of about 600 to about 3,500. Mixtures of two or more polymeric glycol or copolymers can be included.

Examples of polyether glycols that can be used include those glycols with two terminal hydroxy groups, from ring-opening polymerization and/or copolymerization of ethylene oxide, propylene oxide, trimethylene oxide, tetrahydrofuran, and 3-methyltetrahydrofuran, or from condensation.

Polymerization of a polyhydrolic alcohol, such as a diol or diol mixtures, with less than 12 carbon atoms in each molecule, such as ethylene glycol, 1,3-propanediol, 1,4-butanediol, 1,5-pentanediol 1,6-hexanediol, 2,2-dimethyl-1,3 propanediol, 3-methyl-1,5-pentanediol, 1,7-heptanediol, 1,8-octanediol, 1,9-nonanediol, 1,10-decanediol and 1,12-dodecanediol. A linear, bifunctional polyether polyol is preferred, and a poly(tetramethylene ether) glycol with umber average molecular weight of about 1,700 to about 2,100, such as Terathane® 1800 (INVISTA of Wichita, Kans.) with a functionality of 2, is one example of the specific suitable glycols. Co-polymers can include poly(tetramethylene ether co-ethylene ether) glycol and poly(2-methyl tetramethylene ether co-tetramethyleneether) glycol.

Examples of polyester glycols that can be used include those ester glycols with two terminal hydroxy groups, produced by condensation polymerization of aliphatic polycarboxylic acids and polyols, or their mixtures, of low molecular weights with no more than 12 carbon atoms in each molecule. Examples of suitable polycarboxylic acids are malonic acid, succinic acid, glutaric acid, adipic acid, pimelic acid, suberic acid, azelaic acid, sebacic acid, undecanedicarboxylic acid, and dodecanedicarboxylic acid. Examples of suitable glycols for preparing the polyester polyols are ethylene glycol, 1,3-propanediol, 1,4-butanediol, 1,5-pentanediol 1,6-hexanediol, neopentyl glycol, 3-methyl-1,5-pentanediol, 1,7-heptanediol, 1,8-octanediol, 1,9-nonanediol, 1,10-decanediol and 1,12 dodecanediol. A linear bifunctional polyester polyol with a melting temperature of about 5° C. to about 50° C. is an example of a specific polyester glycol.

Examples of polycarbonate glycols that can be used include those carbonate glycols with two terminal hydroxyl groups, produced by condensation polymerization of phosgene, chloroformic acid ester, dialkyl carbonate or diallyl carbonate and aliphatic polyols, or their mixtures, of low molecular weights with no more than 12 carbon atoms in each molecule. Examples of suitable polyols for preparing the polycarbonate polyols are diethylene glycol, 1,3-propanediol, 1,4-butanediol, 1,5-pentanediol, 1,6-hexanediol, neopentyl glycol, 3-methyl-1,5-pentanediol, 1,7-heptanediol, 1,8-octanediol, 1,9-nonanediol, 1,10-decanediol and 1,12-dodecanediol. A linear, bifunctional polycarbonate polyol with a melting temperature of about 5° C. to about 50° C. is an example of a specific polycarbonate polyol.

The diisocyanate component used to make the polyurethaneurea can include a single diisocyanate or a mixture of different diisocyanates including an isomer mixture of diphenylmethane diisocyanate (MDI) containing 4,4′-methylene bis(phenyl isocyanate) and 2,4′-methylene bis(phenyl isocyanate). Any suitable aromatic or aliphatic diisocyanate can be included. Examples of diisocyanates that can be used include, but are not limited to 4,4′-methylene bis(phenyl isocyanate), 4,4′-methylenebis(cyclohexyl isocyanate), 1,4-xylenediisocyanate, 2,6-toluenediisocyanate, 2,4-toluenediisocyanate, and mixtures thereof. Examples of specific polyisocyanate components include Takenate® 500 (Mitsui Chemicals), Mondur® MB (Bayer), Lupranate® M (BASF), and Isonat® 125 MDR (Dow Chemical), and combinations thereof.

Examples of suitable diamine chain extenders for making the polyurethaneurea include: 1,2-ethylenediamine; 1,4-butanediamine; 1,2-butanediamine; 1,3-butanediamine; 1,3-diamino-2,2-dimethylbutane; 1,6-hexamethylenediamine; 1,12-dodecanediamine; 1,2-propanediamine; 1,3-propanediamine; 2-methyl-1,5-pentanediamine; 1-amino-3,3,5-trimethyl-5-aminomethylcyclohexane; 2,4-diamino-1-methylcyclohexane; N-methylamino-bis(3-propylamine); 1,2-cyclohexanediamine; 1,4-cyclohexanediamine; 4,4′-methylene-bis(cyclohexylamine); isophorone diamine; 2,2-dimethyl-1,3-propanediamine; meta-tetramethylxylenediamine; 1,3-diamino-4-methylcyclohexane; 1,3-cyclohexanediamine; 1,1-methylene-bis(4,4′-diaminohexane); 3-aminomethyl-3,5,5-trimethylcyclohexane; 1,3-pentanediamine(1,3-diaminopentane); m-xylylene diamine; and Jeffamine® (Texaco). Optionally, water and tertiary alcohols such as tert-butyl alcohol and u-Cumyl alcohol can also be used as chain extenders to make the polyurethaneurea.

When a polyurethane is desired, a chain extender or mixture of chain extenders used should be a diol. Examples of such dials that may be used include, but are not limited to, ethylene glycol, 1,3-propanediol, 1,2-propylene glycol, 3-methyl-1,5-pentanediol, 2,2-dimethyl-1,3-trimethylene diol, 2,2,4-trimethyl-1,5-pentanediol, 2-methyl-2-ethyl-1,3-propanediol, 1,4-bis(hydroxyethoxy)benzene, 1,4-butanediol, and mixtures thereof.

A monofunctional alcohol or a primary/secondary monofunctional amine can be included as a chain terminator to control the molecular weight of the polyurethaneurea. Blends of one or more monofunctional alcohols with one or more monofunctional amines may also be included.

Examples of monofunctional alcohols useful as a chain terminator with the present invention include at least one member selected from the group consisting of aliphatic and cycloaliphatic primary and secondary alcohols with 1 to 18 carbons, phenol, substituted phenols, ethoxylated alkyl phenols and ethoxylated fatty alcohols with molecular weight less than about 750, including molecular weight less than 500, hydroxyamines, hydroxymethyl and hydroxyethyl substituted tertiary amines, hydxoxymethyl and hydroxyethyl substituted heterocyclic compounds, and combinations thereof; including furfuryl alcohol, tetrahydrofurfuryl alcohol, N-(2-hydroxyethyl)succinimide, 4-(2-hydroxyethyl)morpholine, methanol, ethanol, butanol, neopentyl alcohol, hexanol, cyclohexanol, cyclohexanemethanol, benzyl alcohol, octanol, octadecanol, N,N-diethylhydxoxylamine, 2-(diethylamino) ethanol, 2-dimethylaminoethanol, and 4-piperidineethanol, and combinations thereof. Preferably, such a monofunctional alcohol is reacted in the step of making the urethane prepolymer to control the polymer molecular weight of polyurethaneurea formed at a later step.

Examples of suitable monofunctional primary amines useful as a chain terminator for the polyurethaneurea include, but are limited to, ethylamine, propylamine, isopropylamine, n-butylamine, sec-butylamine, tert-butylamine, isopentylamine, hexylamine, octylamine, ethylhextylamine, tridecylamine, cyclohexylamine, oleylamine and stearylamine. Examples of suitable monofunctional dialkylamine chain blocking agents include: N,N-diethylamine, N-ethyl-N-propylamine, N,N-diisopropylamine, N-tert-butyl-N-methylamine, N-tert-butyl-N-benzylamine, N,N-dicyclohexylamine, N-ethyl-N-isopropylamine, N-tertbutyl-N-isopropylamine, N-isopropyl-N-cyclohexylamine, N-ethyl-N-cyclohexylamine, N,N-diethanolamine, and 2,2,6,6-tetramethylpiperidine. Preferably, such a monofunctional amine is used during the chain extension step to control the polymer molecular weight of the polyurethaneurea. Optionally, amino-alcohols such as ethanolamine, 3-amino-1-propanol, isopropanolamine and N-methylethanolamine can also be used to regulate the polymer molecular weight during the chain extension reaction.

Classes of additives that may be optionally included in the elastomeric fiber are listed below. An exemplary and non-limiting list is included. However, additional additives are well-known in the art. Examples include: antioxidants, UV stabilizers, colorants, pigments, cross-linking agents, phase change materials (paraffin wax), antimicrobials, minerals (i.e., copper), microencapsulated additives (i.e., aloe vera, vitamin E gel, aloe vera, sea kelp, nicotine, caffeine, scents or aromas), nanoparticles (i.e., silica or carbon), calcium carbonate, flame retardants, antitack additives, chlorine degradation resistant additives, vitamins, medicines, fragrances, electrically conductive additives, dyeability and/or dye-assist agents (such as quaternary ammonium salts).

Other additives which may be added to the include adhesion promoters and fusibility improvement additives, anti-static agents, anti-creep agents, optical brighteners, coalescing agents, electroconductive additives, luminescent additives, lubricants, organic and inorganic fillers, preservatives, texturizing agents, thermochromic additives, insect repellants, and wetting agents, stabilizers (hindered phenols, zinc oxide, hindered amine), slip agents (silicone oil) and combinations thereof.

The additive may provide one or more beneficial properties including: dyeability, hydrophobicity (i.e., polytetrafluoroethylene (PTFE)), hydxophilicity (i.e., cellulose), friction control, chlorine resistance, degradation resistance (i.e., antioxidants), adhesiveness and/or fusibility (i.e., adhesives and adhesion promoters), flame retardance, antimicrobial behavior (silver, copper, ammonium salt), barrier, electrical conductivity (carbon black), tensile properties, color, luminescence, recyclability, biodegradability, fragrance, tack control (i.e., metal stearates), tactile properties, set-ability, thermal regulation (i.e., phase change materials), nutraceutical, delustrant such as titanium dioxide, stabilizers such as hydrotalcite, a mixture of huntite and hydromagnesite, UV screeners, and combinations thereof.

Additives may be included in any amount suitable to achieve the desired effect.

Spandex fibers can be formed from the polyurethane orpolyurethaneurea polymer solution through fiber spinning processes such as dry spinning, wet spinning, or melt spinning. In dry spinning, a polymer solution comprising a polymer and solvent is metered through spinneret orifices into a spin chamber to form a filament or filaments. Polyurethaneureas are typically dry-spun or wet-spun when spandex fibers made therefrom are desired. Polyurethanes are typically melt-spun when spandex fibers made therefrom are desired.

Typically, a polyurethaneurea polymer is dry spun into filaments from the same solvent as has been used for the polymerization reaction. Gas is passed through the chamber to evaporate the solvent to solidify the filament(s). Filaments are dry spun at a windup speed of at least 200 meters per minute. The spandex can be spun at a speed at any desired speed such as in excess of 800 meters/minute. As used herein, the term “spinning speed” refers to the yarn take-up speed.

Good spinability of spandex filaments is characterized by infrequent filament breaks in the spinning cell and in the wind up. The spandex can be spun as single filaments or can be coalesced by conventional techniques into multi-filament yarns. Each filament in multifilament yarn can typically be of textile decitex (dtex), e.g., in the range of 6 to 25 dtex per filament.

Spandex in the form of a single filament or a multifilament yarn is typically used for elasticizing substrates to form the composite structures herein. Multifilament spandex yarn frequently will comprise from about 4 to about 120 filaments per strand of yarn. Spandex filaments or yarns which are especially suitable are those ranging from about 200 to about 3600 decitex, including from about 200 decitex to about 2400 decitex and from about 540 to about 1880 decitex.

The inner layer of elastomeric fiber is adhesively bonded or attached to the relatively inelastic substrates being elasticized. Adhesive bonding of the selected type of polyurethane herein to such inelastic flexible substrates is generally brought about through the use of a conventional hot melt adhesive.

Conventional hot melt adhesives are typically thermoplastic polymers which exhibit high initial tack, provide good bond strength between the components and have good ultraviolet and thermal stability. Preferred hot melt adhesives will be pressure sensitive. Examples of suitable hot melt adhesives am those comprising a polymer selected from the group consisting of styrene-isoprene-styrene (SIS) copolymers; styrene-butadiene-styrene (SBS) copolymers; styrene-ethylene-butylene-styrene (SEBS) copolymers; ethylene-vinyl acetate (EVA) copolymers; amorphous poly-alpha-olefin (APAO) polymers and copolymers; and ethylene-styrene interpolymers (ESI). Most preferred are adhesives based on styrene-isoprene-styrene (SIS) block copolymers. Hot melt adhesives are commercially available.

They are marketed under designations such as H-2104, H-2494, H-4232 and H-20043 from Bostik; HL-1486 and HL-1470 from H.B. Fuller Company; and NS-34-3260, NS-34-3322 and NS-34-560 from National Starch Company.

The present invention also provides a process for manufacturing these stretch nonwoven or elasticized fabric composites.

The process comprises placing between two layers of nonwoven or fabric an inner layer of elastomeric fibers with multiple ends arranged in close spacing. In one nonlimiting embodiment, the inner layer is under tension. In one nonlimiting embodiment, the inner layer is drafted 2× to 4×. In one nonlimiting embodiment, the inner layer is drafted 2.5× to 4×. In one nonlimiting embodiment of this process, the inner layer of elastomeric fibers comprises 10-700 ends. In one nonlimiting embodiment of this process, the elastomeric fiber of the inner layer is spaced 1.5 mm-5 mm apart.

The two layers of nonwoven or fabric and the inner layer of elastomeric fibers are then bonded by applying an adhesive composition. In one nonlimiting embodiment, the adhesive is applied to the inner layer fibers and attached to the nonwoven. In one nonlimiting embodiment, the nonwoven is free from adhesive.

Glue migration through the porous nonwoven or fabric will result in excessive downtime to clean the glue buildup laminator. Furthermore, glue migration into the web will result in sticky and harsh hand feel of the nonwoven or fabric. Accordingly, preferred is that web integrity or fiber bonding integrity to the nonwoven or fabric be arranged to stop or minimize glue migration into the nonwoven or fabric.

In one nonlimiting embodiment, a beam arranged fiber feeding system is used to feed the inner layer of elastomeric fiber and adhesive onto the top and/or bottom nonwoven or fabric outer layers.

In another nonlimiting embodiment, a multi creel fiber arranged system is used to feed the inner layer of elastomeric fiber and adhesive is applied to the inner layer fiber before attaching onto the top and/or bottom nonwoven or fabric outer layers. The creel system allows the feed of 10-200 ends without compromising fiber or web integrity.

In one nonlimiting embodiment, a chilled roll is used in the process to quench the hot temperature of the adhesive thereby stopping or minimizing migration of the adhesive into the nonwoven or fabric substrate.

Also provided by the present invention are articles of manufacture, at least portion of which comprises the stretch nonwoven or elasticized fabric composite disclosed herein. Nonlimiting examples of such articles of manufacture include home textiles, medical components, personal hygiene articles, diapers, adult incontinence garments and bandages. Articles of manufacture prepared with the stretch nonwoven or elasticized fabric composite disclosed herein have better hand feel, fit and comfort.

All patents, patent applications, test procedures, priority documents, articles, publications, manuals, and other documents cited herein are fully incorporated by reference to the extent such disclosure is not inconsistent with this invention and for all jurisdictions in which such incorporation is permitted.

The following Test Method demonstrates the present invention and its capability for use. The invention is capable of other and different embodiments, and its several details are capable of modifications in various apparent respects, without departing from the scope and spirit of the present invention. Accordingly, the Test Method is to be regarded as illustrative in nature and non-limiting.

### Test Method for Composites

A test methodology used to test retractive force of composite is tensile testing using ASTM D4964.

1. A stretch nonwoven or elasticized fabric composite comprising: (a) two outer layers of nonwoven or fabric of substantially equal width wherein each layer has an inside surface and an outside surface with respect to the composite fabric; (b) an inner layer of elastomeric fiber with multiple ends arranged in close spacing; and (c) an adhesive composition bonding the outer and inner layers.

2. The stretch nonwoven or elasticized composite fabric of claim 1 wherein the inner layer is under tension.

3. The stretch nonwoven or elasticized composite fabric of claim 1 wherein the inner layer is drafted 2× to 4×.

4. The stretch nonwoven or elasticized composite fabric of claim 1 wherein the inner layer is drafted 2.5× to 4×.

5. The stretch nonwoven or elasticized composite fabric of claim 1 wherein said inner layer of elastomeric fiber comprises 10-700 ends.

6. The stretch nonwoven or elasticized composite fabric of claim 1 wherein said elastomeric fiber of said inner layer is spaced 1.5 mm-5 mm apart.

7. The stretch nonwoven or elasticized composite fabric of claim 1 wherein the elastomeric fiber comprises spandex.

8. A process for manufacturing a stretch nonwoven or elasticized fabric composite comprising the steps of:
(a) placing between top and bottom outer layers of nonwoven or fabric an inner layer of elastomeric fiber with multiple ends arranged in close spacing; and
(b) bonding the top and bottom outer layers of nonwoven or fabric and the inner layer of elastomeric fiber by applying an adhesive composition.

9. The process of claim 8 wherein the inner layer is under tension.

10. The process of claim 8 wherein the inner layer is drafted 2× to 4×.

11. The process of claim 8 wherein the inner layer is drafted 2.5× to 4×.

12. The process of claim 8 wherein said inner layer of elastomeric fibers comprises 10-700 ends.

13. The process claim 8 wherein said elastomeric fiber of said inner layer is spaced 1.5 mm-5 mm apart.

14. The process of claim 8 wherein the elastomeric fiber comprises spandex.

15. The process of claim 8 wherein a beam arranged fiber feeding system feeds the inner layer of elastomeric fiber and adhesive onto the top and/or bottom nonwoven or fabric outer layers.

16. The process of claim 8 wherein a multi creel fiber arranged system feeds the inner layer of elastomeric fiber onto the top and/or bottom nonwoven or fabric outer layers.

17. The process of claim 16 wherein the creel system feeds 10-200 ends.

18. The process of claim 8 wherein the adhesive is hot melt adhesive and a chilled roll quenches the hot temperature of the adhesive to stop or minimize migration into the nonwoven or fabric layers.

19. An article of manufacture at least a portion of which comprises the stretch nonwoven or elasticized fabric composite of claim 1.

20. The article of manufacture of claim 19 which comprises a home textile, a medical component, a personal hygiene article, a diaper, an adult incontinence garment or a bandage.
